(19) United States
(12) Patent Application Publication
(10) Pub. No.: US 2026/0041910 A1
(43) Pub. Date: Feb. 12, 2026

(54) A VAGUS NERVE STIMULATION APPARATUS

(71) Applicant: MindSpire Ltd., Tunbridge Wells (GB)

(72) Inventor: Jane Ollis, Robertsbridge (GB)

(21) Appl. No.: 19/138,682

(22) PCT Filed: May 28, 2024
(86) PCT No.: PCT/GB2024/051361

§ 371 Date: Jun. 13, 2025

(30) Foreign Application Priority Data
May 25, 2023 (GB) 2307877.7

(51) Int. Cl. A61N 1/36; A61M 21/00; A61M 21/02; A61N 1/04

(52) U.S. Cl. CPC A61N 1/36031; A61M 21/02; A61N 1/0456; A61N 1/0476; A61N 1/0484; A61N 1/36021; A61M 2021/0027; A61M 2021/0072

(57) ABSTRACT

A non-invasive vagus nerve stimulation apparatus comprising a pair of headphones, a vagus nerve stimulator; one or more cardiac and/or brain activity sensors; and a controller; wherein the pair of headphones includes a pair of spaced apart ear-covering portions and a headband which connects the spaced apart ear-covering portions; the vagus nerve stimulator is carried by one of the ear-covering portions and includes a first arm which carries a cathode and a second arm which carries an anode; the or each cardiac and/or brain activity sensor senses activity within the body of a user and signals relating to the cardiac and/or brain activity are transmitted from the or each electrical activity sensor to the controller; and wherein the controller is carried by the pair of headphones, processes the signals relating to the sensed cardiac and/or brain activity and controls the vagus nerve stimulator to electrically stimulate the vagus nerve.

### CROSS-REFERENCE TO RELATED APPLICATIONS

This application is a United States National Phase Entry of PCT Appl. No. PCT/GB2024/051361 titled, “A VAGUS NERVE STIMULATION APPARATUS” filed on May 28, 2024, which claims priority to Great Britain Appl. No. GB2307877.7, titled, “A VAGUS NERVE STIMULATION APPARATUS” filed on May 25, 2023.

### BACKGROUND OF THE INVENTION

### Field of the Art

The present invention relates to a vagus nerve stimulation apparatus. In particular, it relates to an apparatus which can measure electrical activity within the body and determine appropriate stimulation of the vagus nerve to treat conditions associated with an imbalance between the sympathetic and parasympathetic nervous systems, such as, for example, chronic stress and anxiety, tinnitus, hypertension, pain and neurological degeneration. It may also be used in stroke rehabilitation.

### Discussion of the State of the Art

The treatment of stress-related illnesses with drugs is not a good solution to the problem as the average efficacy rate of most drugs does not exceed 50%. Additionally, drug-based treatments tend to be associated with intolerable adverse side effects. To address these issues and minimize the adverse side effects of drugs, alternate treatments have been sought in recent years. Such alternate methods include treatments based on nerve stimulation, which is sometimes referred to as neuromodulation.

### BRIEF DESCRIPTION OF THE DRAWING FIGURES

The accompanying drawings illustrate several embodiments of the invention.

FIG. 1 is a perspective view of a non-invasive vagus nerve stimulation apparatus according to the first aspect of the invention;

FIG. 2 is a side elevational view of the stimulator/sensor arrangement which forms part of the apparatus shown in FIG. 1; and

FIG. 3 is a schematic representation of the stimulator/sensor arrangement shown in FIG. 2.

### DETAILED DESCRIPTION

Vagus nerve stimulation has been investigated since 1990 using implanted or invasive VNS (vagus nerve stimulation) devices.

The vagus nerve is the tenth cranial nerve and is considered to be the major parasympathetic innervation of the autonomic nervous system.

In order to avoid invasive procedures, non-invasive methods of VNS have been explored.

It is known that the vagus nerve has a branch that is located adjacent to the ear. This is often referred to as the auricular branch of the vagus nerve (ABVN) but is also known as Alderman's nerve or Arnold's nerve. In the context of the present invention, reference to stimulation of the vagus nerve should be interpreted as stimulation of the auricular branch of the vagus nerve.

The electrical stimulation of the auricular branch of the vagus nerve may be achieved via the tragus or the cymba conchae, as these areas include the most vagus fibres.

The vagus nerve has also been associated with other conditions, such as tinnitus, hypertension, pain, neurological degeneration and stroke rehabilitation. Accordingly, stimulation of the vagus nerve may be used to treat or as part of a treatment for tinnitus, hypertension, pain (e.g., chronic pain), stroke rehabilitation, neurological degeneration and stress-related conditions (such as anxiety and chronic stress).

According to a first aspect of the invention, there is provided a non-invasive vagus nerve stimulation apparatus for use in the treatment of stress and/or anxiety, wherein the apparatus includes a pair of headphones, a vagus nerve stimulator; one or more cardiac and/or brain activity sensors; and a controller; wherein the pair of headphones includes a pair of spaced apart ear-covering portions and a headband which connects the spaced apart ear-covering portions; the vagus nerve stimulator is carried by one or both of the ear-covering portions and includes a first arm which carries a cathode and a second arm which carries an anode; the or each cardiac and/or brain activity sensor senses cardiac and/or brain activity within the body of a user and signals relating to the cardiac and/or brain activity are transmitted from the or each cardiac and/or brain activity sensor to the controller; and wherein the controller is carried by the pair of headphones, processes the signals relating to the sensed cardiac and/or brain activity and controls the vagus nerve stimulator to electrically stimulate the vagus nerve.

The controller suitably controls the vagus nerve stimulator in response to the sensed signal(s) from the cardiac and/or brain activity sensor(s).

The invention thus provides an apparatus for stimulating the ABVN, optionally in response to sensed cardiac and/or brain activity within a patient's body. Thus, when a patient is stressed or is anxious for example, the elevated heart activity and/or brain electrical activity that results from this condition may be sensed and the vagus nerve may be stimulated to reduce the stress and/or anxiety levels. This results from the stimulation of the parasympathetic nervous system.

It will be appreciated that the sensed cardiac and/or brain activity may function as a biofeedback mechanism and the stimulation of the vagus nerve may be controlled in response to the sensed cardiac and/or brain activity.

In the context of the present invention, the term “non-invasive” means that the apparatus is not surgically implanted within the body of the patient. Thus, it may be readily worn by and removed from the patient without the need for specialist medical intervention.

It will be appreciated that the cardiac and/or brain activity that is sensed by the cardiac and/or brain activity sensor(s) is suitably the activity of the heart (e.g. via ECG sensing or PPG sensing) and/or the brain (e.g. via EEG sensing). It will be appreciated that sensors relating to brain activity may measure the brain's electrical activity. Similarly, sensors relating to heart activity may measure the electrical activity of the heart (e.g., vie ECG sensing) or measure blood flow data (e.g., via PPG sensing). Thus, the cardiac and/or brain sensor may be selected from an EEG sensor, an ECG sensor and a PPG sensor.

As noted above, imbalances between the sympathetic nervous system and the parasympathetic nervous system may result in increased activity in the heart and/or brain. This increased activity may be measured and used in the control of the vagus nerve stimulation.

It has been found that activity of the heart/brain may be sensed by a sensor located adjacent to the mastoid bone, for example, or within a portion of the ear canal.

The mastoid bone is located behind the ear, whereas the stimulator needs to be located on or within the ear. Accordingly, the apparatus may include both the cardiac and/or brain activity sensor and the vagus nerve stimulator. Optionally, the apparatus may also include a connector which connects the or each cardiac and/or brain activity sensor and the vagus nerve stimulator. Thus, one of or both of the ear-covering portions may include a connector which connects the or each cardiac and/or brain activity sensor and the vagus nerve stimulator. Alternatively, the sensor and the stimulator may be embedded in one or both of the ear-covering portions of the apparatus. In embodiments in which the cardiac and/or brain activity sensor is located adjacent to the mastoid bone, the connector may orient or position the relative locations of the cardiac and/or brain activity sensor and the vagus nerve stimulator. For example, the connector may be an arm which locates and retains the cardiac and/or brain activity sensor and the vagus nerve stimulator in the desired locations in use. In certain embodiments of the invention, the arm may be resiliently deflectable. In such embodiments, the arm may bias the cardiac and/or brain activity sensor and the electrode stimulator to their desired locations.

In the embodiment discussed above, the cardiac and/or brain activity sensor or one of the cardiac and/or brain activity sensors and the vagus nerve stimulator may form part of a single component, i.e., a component (e.g., an ear-covering portion of the apparatus) which includes both the sensor and the stimulator. However, in an alternative embodiment the vagus nerve stimulator is carried by a first ear-covering portion, and the or one of the cardiac and/or brain activity sensors is carried by the second ear-covering portion. Accordingly, at least one of the cardiac and/or brain activity sensors may be carried by one of the ear-covering portions of the headphones.

In further embodiments of the invention, the cardiac and/or brain activity sensor(s) may be in the form of electrodes that are attached to pre-determined positions of a patient's body. In other words, the cardiac and/or brain activity sensor(s) may be separate from the headphones.

The headband of the headphones may adjustable such that the ear-covering portions may be positioned correctly relative to the patient.

The controller may be carried by the headband of the headphones, or it may be carried by one of the ear-covering portions of the headphones.

In an embodiment of the invention, the cardiac and/or brain activity sensor(s) sense ECG signals, PPG signals and/or EEG signals. It is well known that the activity of the heart (measured by ECG sensors and/or PPG sensors) and/or the electrical activity of the brain (measured by EEG sensors) can be used as an indicator or biomarker for imbalances in the sympathetic and parasympathetic nervous systems, such as in chronic stress and/or anxiety conditions. Other indications, such as tinnitus, hypertension, pain and neurological degeneration may also result in corresponding activity within the body, which may be sensed by the cardiac and/or brain activity sensor(s). Accordingly, an indication of the imbalance (and therefore an indication of the level of stress and/or anxiety within the patient, for example) can be measured via measurement of the activity of the heart and/or the brain. This in turn may be used in a biofeedback loop to control the stimulation of the vagus nerve.

Suitably, at least one of the cardiac and/or brain activity sensors measures heart rate and the sensor is a PPG sensor. PPG sensors may be located behind the ear, i.e., behind the helix of the ear. Accordingly, in embodiments in which a PPG sensor is carried by one of the ear-covering portions of the headphones, it may be arranged to be located behind the ear of a patient.

As noted above, two possible access points to the auricular branch of the vagus nerve are the tragus and the cymba conchae. The tragus is more easily accessed, but the cymba conchae has a greater portion of vagus nerve fibres.

Thus, the anode and/or the cathode of the vagus nerve stimulator may be located adjacent to or attached to the tragus. Suitably, the or each electrode of the stimulator contacts the tragus. For example, the vagus nerve stimulator may include a pair of legs which are biased towards each other, wherein one or both of the legs includes a stimulator electrode (i.e., a cathode or anode). In such an embodiment, the stimulator may be in the form of a clip, wherein the legs are hinged at one end (a proximal end) and the distal ends of the legs are biased towards each other. In this way, the clip may be attached to the tragus with each leg being disposed on a respective side of the tragus. One or both of the legs may include a stimulator electrode.

Alternatively, one or both of the electrodes of the stimulator may be located adjacent to the cymba conchae. Again, the or each electrode may contact the cymba conchae. In such embodiments, the electrode(s) may be retained in the desired position by a locating element which forms a part of one of the ear-covering portions or by the connector.

In a further embodiment of the invention, one of the cathode and anode of the vagus nerve stimulator is adjacent to the tragus and the other of the cathode and anode is adjacent to the cymba conchae in use. Thus, the headphones may include a first locating element which locates one of the cathode and anode of the stimulator adjacent to the tragus, and a second locating element which locates the other of the cathode and the anode adjacent to the cymba conchae.

As noted above, in order to retain in the desired positions, the cardiac and/or brain activity sensor and the vagus nerve stimulator, the connector or locating element may comprise an arm, which in turn may be resiliently deflectable. Accordingly, the arm may be deflected from a rest configuration to an in-use configuration. In the “in-use” configuration, the arm may exert a biasing force on the cardiac and/or brain activity sensor and/or the nerve stimulator. The biasing force suitably retains the respective components in their desired positions.

Alternatively, one of the ear-covering portions of the headphones may include a locating element which carries the sensor or the stimulator. For example, the ear-covering portion may include a first locating element which carries the (or one of the) sensor and a second locating element which carries the stimulator. Alternatively, one of the ear-covering portions includes a first locating element that carries the (or one of the) cardiac and/or brain activity sensor, and the other ear-covering portion includes a second locating element that carries the vagus nerve stimulator.

The or each ear-covering portion may include a padded portion which is formed from a resiliently deformable polymeric material, e.g., a foamed material. The padded portion may make the headphones more comfortable to wear.

In an embodiment of the invention, the apparatus further includes a power source. The power source suitably powers the or each cardiac and/or brain activity sensor and the vagus nerve stimulator. The power source may be a rechargeable battery, or it may be disposable battery. Suitably, the battery is carried by the headphones. In embodiments in which the power source is a rechargeable battery, the battery may be disposed within the headphones. In such embodiments, the headphones may include a port via which the battery may be charged.

Suitably, the controller is carried by the headphones. For example, the controller may be disposed within the headband or one of the ear-covering portions. The connection between the controller and the or each cardiac and/or brain activity sensor may be a wired connection or a wireless connection. Similarly, the connection between the controller and the vagus nerve stimulator may be a wired connection or a wireless connection.

It is known to determine a respiration (inhalation/exhalation) cycle based on associated activity of the body, for example heart rate. Accordingly, it is possible to determine the respiration cycle of a user via the or one of the cardiac and/or brain activity sensors. In an embodiment of the invention, the controller controls the vagus nerve stimulator such that the vagus nerve is only stimulated during exhalation of the user/patient. Without wishing to be bound by theory, it has been noted that stimulation of the vagus nerve only during exhalation may improve outcome.

It is known that music and certain other sounds may have a soothing or calming effect on a patient, which in turn may reduce the symptoms experienced by the patient, such as for example chronic stress and/or anxiety. Additionally, certain sounds may be useful in the treatment of tinnitus. In view of this, the or each ear-covering portion may include a speaker through which sounds may be transmitted into the ear of the patient when wearing the headphones.

It may be desirable to record data received by the controller from the or each cardiac and/or brain activity sensor and/or data relating to the operation of the vagus nerve stimulator. Accordingly, the apparatus may include a transmitter which transmits data relating to the or each cardiac and/or brain activity sensor and/or the vagus nerve stimulator to a remote device, such as a server or other computing device, such as a smartphone. The transmitter may be connected to the remote device via a wired connection or via a wireless connection (e.g., via Bluetooth™). As the controller receives signals from the cardiac and/or brain activity sensor(s) and transmits control signals to the stimulator, the transmitter may form part of the controller.

According to a second aspect of the invention, there is provided a method of stimulating the parasympathetic nervous system, wherein the method includes providing an apparatus according to the first aspect of the invention as defined anywhere herein; attaching the pair of headphones to a patient such that each ear-covering portion covers a respective ear of the patient; locating one or more cardiac and/or brain activity sensors adjacent to one or more portions of the patient's body where activity signals may be sensed; locating the anode and cathode of the or each vagus nerve stimulator adjacent to respective portions of the auricular vagus nerve in a respective ear of the patient; sensing signals relating to cardiac and/or brain activity via the or each activity sensor; processing the signals; determining an appropriate stimulation of the vagus nerve from the sensed cardiac and/or brain signals; and controlling the vagus nerve stimulator to stimulate the auricular vagus nerve.

As noted above, an imbalance in the sympathetic nervous system and the parasympathetic nervous system may be present in patients who are suffering from tinnitus, hypertension, pain, neurological degeneration and stress-related conditions, or who are undergoing stroke rehabilitation. Accordingly, the method of the second aspect of the invention may be used to treat tinnitus, hypertension, pain, neurological degeneration and stress-related conditions or to assist with stroke rehabilitation. Suitably, the stress-related condition is anxiety and/or chronic stress. Additionally, pain may be chronic pain.

In the context of the present invention, the term “treat” is used to indicate a reduction in the symptoms of the condition and/or the underlying cause of the condition.

As noted above, the cardiac and/or brain activity sensor suitably senses the activity of the heart and/or brain. For example, the activity sensor may sense EEG, PPG and/or ECG signals. Accordingly, the step of sensing cardiac and/or brain signals within the body may include sensing the activity of the heart and/or brain, for example via ECG, PPG and/or ECG sensors.

In an embodiment of the second aspect of the invention, the step of locating the cathode and/or anode of the vagus nerve stimulator adjacent to a portion of the auricular vagus nerve in the ear includes locating the cathode and/or anode adjacent to the tragus. For example, the anode may be located on one side of the tragus and the cathode may be located on the other side of the tragus. Alternatively, the anode or the cathode may be located only on one side of the tragus.

As an alternative to stimulating the vagus nerve via the tragus, the vagus nerve may be stimulated via the cymba conchae. Thus, the step of locating the cathode and/or anode of the vagus nerve stimulator adjacent to a portion of the auricular vagus nerve in the ear includes locating the cathode and/or anode adjacent to the cymba conchae. Again, the anode may be located on one side of the cymba conchae and the cathode may be located on the other side of the cymba conchae. Alternatively, the anode or the cathode may be located only on one side of the cymba conchae.

In a further embodiment of the second aspect of the invention, the step of locating the cathode and/or anode of the vagus nerve stimulator adjacent to a portion of the auricular vagus nerve in the ear includes locating one of the cathode and anode adjacent to the cymba conchae and locating the other of the cathode and anode adjacent to the tragus.

In a further embodiment of the second aspect of the invention, the step of locating one or more cardiac and/or brain activity sensors adjacent to one or more portions of the patient where activity signals may be sensed may include positioning an cardiac and/or brain activity sensor adjacent to the mastoid bone. Thus, it may comprise positioning the cardiac and/or brain activity sensor behind the ear (i.e., between the ear and the mastoid bone).

In embodiments in which the or each ear-covering portion of the headphones includes a speaker, the method may further comprise the step of transmitting audio signals to the patient, for example music or other sounds or predetermined sound frequencies.

It may be useful for the patient to record the sensed activity levels and optionally also their use of the apparatus to stimulate the vagus nerve. Accordingly, the apparatus may be connected to a remote device which is capable of storing data relating to the sensed cardiac and/or brain activity data of the patient and/or the activation of the vagus nerve stimulator (e.g., the patient's use of the apparatus). The remote device may be a portable device, such as a mobile telephone or other portable computing device, such as a laptop computer or a tablet computer. Alternatively, the remote device may be a remote server. The apparatus may connect to the remote device via a wireless connection. This may be via a local wireless connection, such as a Bluetooth® connection or it may be via a wide area network or a wireless communications network, such as a mobile communications network or the internet. Accordingly, the apparatus may further include a wireless data transmitter.

The skilled person will appreciate that the features described and defined in connection with the aspects of the invention and the embodiments thereof may be combined in any combination, regardless of whether the specific combination is expressly mentioned herein. Thus, all such combinations are considered to be made available to the skilled person.

An embodiment of the invention will now be described, by way of example only, with reference to the accompanying drawings in which:

For the avoidance of doubt, the skilled person will appreciate that in this specification, the terms “up”, “down”, “front”, “rear”, “upper”, “lower”, “width”, etc. refer to the orientation of the components as found in the example when installed for normal use as shown in the Figures.

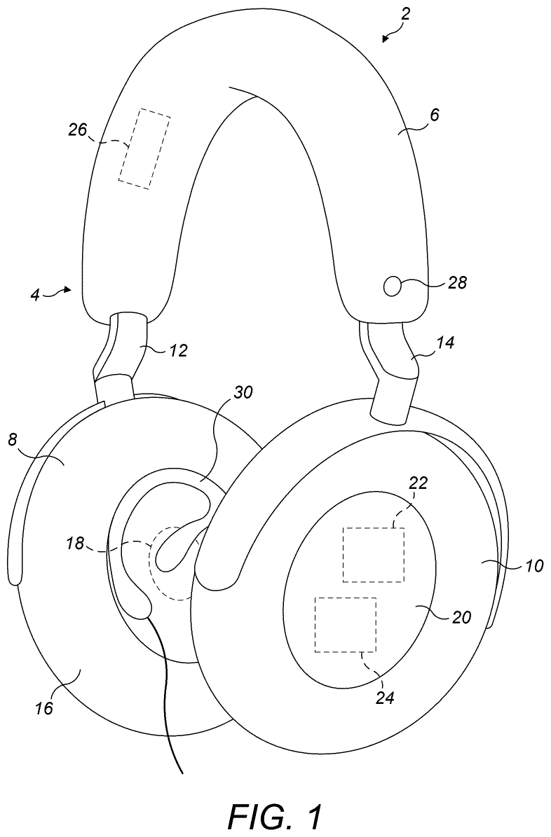

A non-invasive vagus nerve stimulation apparatus 2 is shown in FIG. 1. The apparatus 2 includes a pair of headphones 4 which comprise a headband 6 which connects a first ear-covering portion 8 and a second ear-covering portion 10.

The first ear-covering portion 8 includes an adjustable coupling 12 which connects it to the headband 6. The second ear-covering portion 10 includes a corresponding coupling 14 which connects it to the headband 6. Such adjustable couplings are known in the context of headphones and permit each of the ear-covering portions 8, 10 to be rotated relative to the headband 6 and extended from or retracted towards the headband 6.

Each of the ear-covering portions further includes an annular padded element 16 (only shown in FIG. 1 in connection with the first ear-covering portion 8). Again, such annular padded elements are known in the context of headphones.

Disposed in the middle of each of the ear-covering portions 8, 10 and not covered by the respective padded element 16 is a speaker 18 (again, only shown in FIG. 1 in connection with the first ear-covering portion 8) which is able to emit sound waves.

On the outwardly facing surface of each of the ear-covering portions 8, 10 is a cover 20 (only shown in FIG. 1 in connection with the second ear-covering portion 10). The cover 20 of the second ear-covering portion 10 covers a controller 22 and a wireless Bluetooth® transceiver 24, which will be discussed in more detail below. The skilled person will appreciate that both the controller 22 and the wireless transceiver 24 may be located instead behind the cover of the first ear-covering portion 8, or one of the controller 22 and the wireless transceiver 24 may be located in the first ear-covering portion 8 and the other of the controller 22 and the wireless transceiver 24 may be located in the second ear-covering portion 10.

The headband 6 includes embedded therein a rechargeable battery 26 and a charging port 28.

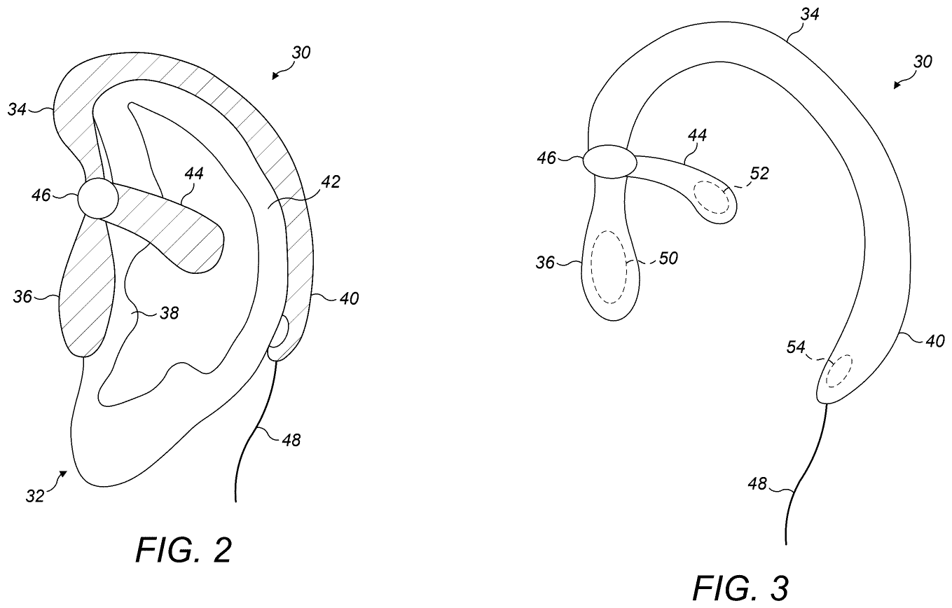

Each of the ear-covering portions 8, 10 further includes a combined stimulator/sensor component 30, which is shown in more detail in FIGS. 2 and 3.

FIG. 2 shows the stimulator/sensor component 30 in position relative to a patient's ear 32. The stimulator/sensor 30 includes a first body member 34 comprising a first end portion 36 that overlies the tragus 38 of the ear 32, and a second end portion 40 which is disposed behind the helix 42 of the ear 32. The second end portion 40 is disposed adjacent the mastoid bone (not shown) of the patient. The first body member 34 is resiliently deformable and is self-supporting relative to the ear 32.

A second body member 44 is hingeably coupled to the first body member 34 via a hinge 46. The second body member 44 overlies a portion of the cymba conchae of the ear 32.

A wire 48 connects the stimulator/sensor component 30 to the controller 22.

FIG. 3 shows a schematic representation of the stimulator/sensor component 30.

The first end portion 36 of the first body member 34 carries a nerve stimulator anode 50. The second body member 44 carries a nerve stimulator cathode 52. The second end portion 40 of the first body member 34 carries a PPG optrode 54.

The signals from the PPG sensor 54 are transmitted via the wire 48 to the controller. Similarly, power to the anode 50 and cathode 52 from the controller is also carried by the wire 48.

The battery 26 carried by the headband 6 powers the speakers 18, the controller 22, the wireless transceiver 24, the anode 50, the cathode 52 and the PPG optrode 54.

In use, the PPG sensor 54 senses heart activity. From these signals, the controller 22 is able to determine an imbalance in the sympathetic nervous system and the parasympathetic nervous system. It is also able to determine the inhalation and exhalation phases of the respiration cycle.

In response to the determined imbalance in the sympathetic nervous system and the parasympathetic nervous systems, the controller activates the anode 50 and cathode 52 to stimulate the vagus nerve. The stimulation of the vagus nerve only occurs during the exhalation phase of the respiration cycle.

In addition to the stimulation of the vagus nerve via the anode 50 and the cathode 52, the controller also controls the emission of sound waves from the speakers 18 carried by the first and second ear-covering portions 8, 10.

The sound waves may be selected for their calming effect on the patient.

Data from the controller 22 in relation to the signals received from the PPG sensor 54 and the activation of the anode 50 and cathode 52 are transmitted wirelessly to a remote server (not shown) by the wireless transceiver 24. The transmitted data is recorded in the respective patient file.

The apparatus 2 therefore includes a biofeedback system in which the desired vagus nerve stimulation is based on heart activity signals received, which in turn indicates a level of stress or other nervous system imbalance experienced by the patient. As the stress levels/imbalance decrease, the vagus nerve stimulation is also decreased.

A proof-of-concept study was carried out and is reported in “Tragus based Vagus Nerve Stimulation for Stress Reduction”; Proceedings of the 14^{th} International Joint Conference on Biomedical Engineering Systems and Technologies (BIOSTEC 2021); Vol 4: BIOSIGNALS; pages 164-168. The study established that stimulation of the vagus nerve via the tragus resulted in a higher parasympathetic vs sympathetic stimulation. The above-noted paper is incorporated herein in its entirety.

1. A non-invasive vagus nerve stimulation apparatus, wherein the apparatus includes a pair of headphones, a vagus nerve stimulator;
one or more cardiac and/or brain activity sensors; and
a controller;
wherein the pair of headphones includes a pair of spaced apart ear-covering portions and a headband which connects the spaced apart ear-covering portions;
the vagus nerve stimulator is carried by one of the ear-covering portions and includes a first arm which carries a cathode and a second arm which carries an anode;
the or each cardiac and/or brain activity sensor senses cardiac and/or brain activity within the body of a user and signals relating to the sensed activity are transmitted from the or each cardiac and/or brain activity sensor to the controller; and
wherein the controller is carried by the pair of headphones, processes the signals relating to the sensed cardiac and/or brain activity and controls the vagus nerve stimulator to electrically stimulate the vagus nerve.

2. The non-invasive vagus nerve stimulation apparatus according to claim 1, wherein the or each cardiac and/or brain activity sensor senses activity of the cardiac system.

3. The non-invasive vagus nerve stimulation apparatus according to claim 1, wherein at least one of the cardiac and/or brain activity sensors is carried by one of the ear-covering portions of the headphones.

4. The non-invasive vagus nerve stimulation apparatus according to claim 3, wherein the activity sensor is a photoplethysmography (PPG) sensor.

5. The non-invasive vagus nerve stimulation apparatus according to claim 4, wherein the PPG sensor is located behind the ear when in use.

6. The non-invasive vagus nerve stimulation apparatus according to claim 1, wherein the cathode and/or anode of the vagus nerve stimulator is adjacent to the tragus in use.

7. The non-invasive vagus nerve stimulation apparatus according to claim 1, wherein the cathode and/or anode of the vagus nerve stimulator is adjacent to the cymba conchae in use.

8. The non-invasive vagus nerve stimulation apparatus according to claim 1, wherein one of the cathode and anode of the vagus nerve stimulator is adjacent to the tragus and the other of the cathode and anode is adjacent to the cymba conchae in use.

9. The non-invasive vagus nerve stimulation apparatus according to claim 1, wherein the apparatus includes a pair of vagus nerve stimulators and each of the vagus nerve stimulators is carried by a respective one of the ear-covering portions.

10. The non-invasive vagus nerve stimulation apparatus according to claim 1, wherein the apparatus senses respiration and the controller only stimulates the vagus nerve during exhalation.

11. The non-invasive vagus nerve stimulation apparatus according to claim 1, wherein the pair of headphones includes a power source.

12. The non-invasive vagus nerve stimulation apparatus according to claim 11, wherein the power source is a battery.

13. The non-invasive vagus nerve stimulation apparatus according to claim 1, wherein each of the ear-covering portions of the pair of headphones includes a respective speaker.

14. The non-invasive vagus nerve stimulation apparatus according to claim 1, wherein the apparatus further includes a wireless transmitter which transmits data relating to the or each cardiac and/or brain activity sensor and/or the vagus nerve stimulator to a remote device.

15. A method of stimulating the parasympathetic nervous system, wherein the method includes providing a non-invasive vagus nerve stimulation apparatus, the method comprising the steps of;
attaching the pair of headphones to a patient such that each ear-covering portion covers a respective ear of the patient;
locating one or more cardiac and/or brain activity sensors adjacent to one or more portions of the patient's body where cardiac and/or brain activity signals may be sensed; locating the anode and cathode of the or each vagus nerve stimulator adjacent to respective portions of the auricular vagus nerve in a respective ear of the patient; sensing signals relating to cardiac and/or brain activity via the or each activity sensor;
processing the signals;
determining an appropriate stimulation of the vagus nerve from the sensed signals; and
controlling the vagus nerve stimulator to stimulate the auricular vagus nerve.

16. The method according to claim 15, wherein the step of sensing cardiac and/or brain activity includes sensing cardiac activity.

17. The method according to claim 15, wherein the step of locating the cathode and/or anode of the vagus nerve stimulator adjacent to a portion of the auricular vagus nerve in the ear includes locating the cathode and/or anode adjacent to the tragus.

18. The method according to claim 15, wherein the step of locating the cathode and/or anode of the vagus nerve stimulator adjacent to a portion of the auricular vagus nerve in the ear includes locating the cathode and/or anode adjacent to the cymba conchae.

19. The method according to claim 15, wherein the step of locating the cathode and/or anode of the vagus nerve stimulator adjacent to a portion of the auricular vagus nerve in the ear includes locating one of the cathode and anode adjacent to the cymba conchae and locating the other of the cathode and anode adjacent to the tragus.

20. The method according to claim 15, wherein the method further includes transmitting sound waves to the patient concurrently with the vagus nerve stimulation.

21-24. (canceled)
